(19) United States
(12) Patent Application Publication
(10) Pub. No.: US 2022/0011298 A1
(43) Pub. Date: Jan. 13, 2022

(54) METHODS AND COMPOSITIONS FOR ISOLATION AND RAPID DETECTION OF MICRO-ORGANISMS FROM BLOOD AND BODILY FLUIDS

(71) Applicant: ACUTIS DIAGNOSTICS, INC., East Northport, NY (US)

(72) Inventors: Mayilvahanan SHANMUGAM, Huntington Station, NY (US); Mumtaz AKHTAR, Bellerose, NY (US); David GOLDBERG, New York, NY (US); Marjorie BON HOMME, Westbury, NY (US)

(21) Appl. No.: 17/370,938

(22) Filed: Jul. 8, 2021

Related U.S. Application Data
(60) Provisional application No. 63/050,509, filed on Jul. 10, 2020.

(51) Int. Cl. G01N 33/50

(52) U.S. Cl. CPC G01N 33/5094

(57) ABSTRACT

The present disclosure provides methods and compositions for testing blood samples to determine the presence and type of a blood stream infection (BSI). In one embodiment, the composition is a lysis reagent or composition that comprises betaine hydrochloride, spermidine, saponin, and Triton® X-100. The methods include combining the lysis reagent with the blood sample, and at least one centrifuge step to isolate the micro-organisms that cause the BSI. The micro-organisms are kept viable so that diagnostic tests can be run on the blood samples after the various method steps are performed.

### CROSS-REFERENCE TO RELATED APPLICATION

The present application claims the benefit of U.S. Provisional Patent Application No. 63/050,509, filed on Jul. 10, 2020, which is herein incorporated by reference.

### BACKGROUND OF THE DISCLOSURE

### 1. Field of the Disclosure

The present disclosure relates to blood test methods and compositions for the rapid determination of the source or cause of a blood stream infection. In particular, the present is disclosure provides a method for rapid determination of the source of infection in a blood stream sample that is inoculated and combined with a novel composition that includes betaine hydrochloride, spermidine, a saponin and a surfactant such as Triton® X-100. The sample is then processed for Gram staining or other diagnostics to determine the type of infection.

### 2. Description of the Related Art

Blood stream infection (BSI) is a worldwide serious medical condition, which leads to life threatening multiple internal organ failure due to dysregulated host response to infection. In the United States, BSI is the predominant cause of in-hospital deaths and annually costs more than US$24 billion.

In healthy patients, blood is sterile. Systemic or localized infections can cause micro-organisms to enter the blood stream, which is known as “bacteremia”. Most of the bacteremia are cleared quickly by the immune system. Overwhelming micro-organism infections can overcome the immune system, resulting in BSI. To identify the micro-organisms responsible for blood stream infection, blood cultures are required. Blood cultures consist of a blood sample from a patient suspected to have a BSI, inoculated into a specialized blood culture bottle containing a liquid broth medium that supports the growth of micro-organisms (bacteria or yeast cells).

In a BSI, the number of micro-organisms per milliliter of patient blood is very low. The detection of microbial growth in blood culture bottles takes several hours (24-72 hours at a minimum) after the blood collected from the patient. Every hour of delay in treatment leads to six to eight percent increase in relative risk of death. Currently, many medical practitioners adopt the practice of “each hour's delay in initiating antibiotics costs lives” and administer antibiotics despite not knowing the extent and type of BSI in the patient. This increases the level of antibiotic resistance due to inappropriate antibiotic administration, which can be a global crisis. Additionally, inappropriate antibiotics or those ill-suited to the type of BSI can also cause harm to the patients, including via organ injury, mitochondrial dysfunction, the impact on the host microbiome, and overgrowth by fungi and Clostridium difficile infection.

To serve the patient in need, better tools and protocols for early diagnosis are a prerequisite for rapid and appropriate antibiotic therapy. Therefore, it is necessary to develop a method for rapid detection of microbial growth from the blood culture bottles. When the growth of micro-organism(s) is detected, a gram stain is done to distinguish gram positive, negative and yeasts. This early information can help clinician determine the most appropriate antibiotic treatment for the patient in need.

Further, in the rapid detection of microbial growth from blood culture bottles, maintaining viability of micro-organisms while removing the blood cells by lysis is critical. The viability of micro-organisms is also important for the downstream testing such as antimicrobial susceptibility testing (AST). Having intact micro-organisms is important for further microbial identification such as PCR or MALDI-TOF mass spectrometry and Next Generation Sequencing (NGS).

### SUMMARY OF THE DISCLOSURE

The methods of the present disclosure enable the isolation of viable micro-organisms from the blood culture bottles immediately after blood collection from the patient, and/or blood culture samples that are already known to be positive for micro-organisms. The methods of the present disclosure include treating the blood culture sample with a composition or lysis reagent that includes a lipotropic agent (for example betaine hydrochloride), a polyamine (for example spermidine), a saponin, and a lysis buffer known to lyse blood cells, such as a non-ionic surfactant (for example Triton® X100). There is also at least one centrifuge step in the method, to concentrate and isolate micro-organisms from the blood sample.

Importantly, the methods of the present disclosure provide viable micro-organisms from just a fraction of the blood culture samples. Rapid microbial growth detection can be conducted on the samples, using time-lapse digital microscopy. The viability of the micro-organisms allows for multiple downstream tests to be performed, such as identification of micro-organisms and AST testing. These methods of the present disclosure also provide an option of preparing and growing a pure culture for further analysis.

In one embodiment, the present disclosure provides a reagent composition for blood lysis solution, comprising a polyamine, a lipotropic agent, a saponin, and a surfactant. The composition can comprise between 0.5 to 1 millimolar of the polyamine, between 0.5 to 1 millimolar of the lipotropic agent, between 0.0909 to 0.2272% by volume of the surfactant, and between 0.2727 to 0.3636% by volume of the saponin.

The present disclosure also provides a method of testing a blood sample of a patient for a blood stream infection that is caused by at least one bacterium. The method comprises the steps of: drawing a sample from the patient; mixing the composition of the preceding paragraph with the sample to form a first mixture; centrifuging the first mixture to separate the first mixture into a supernatant and a pellet; discarding the supernatant; placing the pellet into a growth medium, to form a second mixture; centrifuging the second mixture; and testing the second mixture to determine the presence of the at least one bacterium.

### BRIEF DESCRIPTION OF THE DRAWINGS

FIG. 1 is a schematic depiction of a first method of the present disclosure.

FIG. 2 is a schematic depiction of a second method of the present disclosure.

FIG. 3 is a schematic depiction of a third method of the present disclosure.

FIG. 4 shows digital micrographs at selected time points, confirming the growth of selected micro-organisms after use of the methods of the present disclosure on a blood sample. The micrographs are taken using time lapse digital microscopy.

FIGS. 5a through 5g show growth curves for selected microorganisms as a function of time, where the data is obtained using digital microscopy.

### DETAILED DESCRIPTION OF THE DISCLOSURE

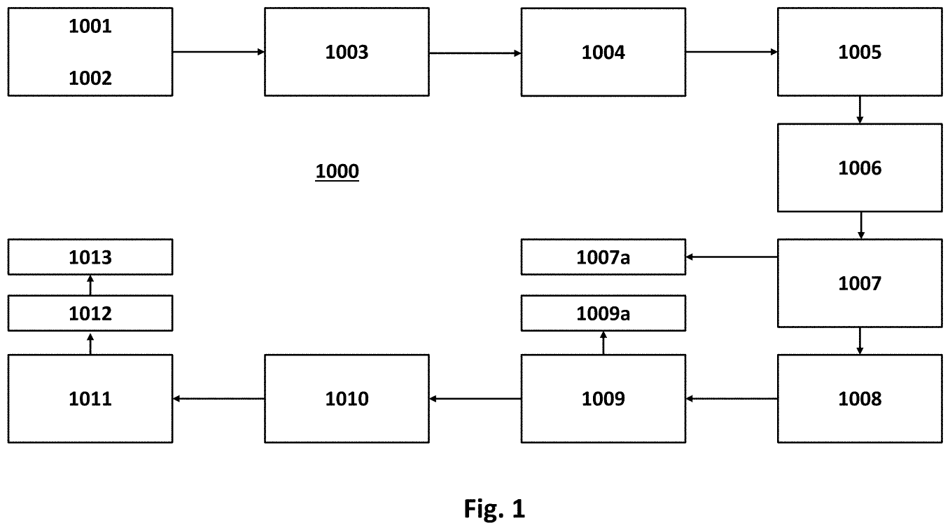

Referring to the Figures, and in particular FIGS. 1-3, schematic drawings of the methods of the present disclosure are shown. The methods of the present disclosure provide for a rapid processing of a freshly inoculated blood sample from a patient to determine if the patient has a blood stream infection (BSI), and if so, what type of bacteria is causing the infection. The methods of the present disclosure can also provide for the rapid analysis of a sample from a patient who is known to have a BSI, but where it is not clear which type. The methods of the present disclosure include treating the blood sample with a novel composition that includes a lipotropic agent, a polyamine, a saponin, and a lysis buffer. In one embodiment, the novel composition includes betaine hydrochloride, spermidine, saponin, and a nonionic surfactant, for example Triton™ X100. The resulting composition is agitated and/or subjected to at least one centrifuge step to separate the components of the composition.

Suitable lipotropic agents include betaine hydrochloride, oxibetaine, trimethlyglycine, inositol, methionine, and any combinations thereof. In one embodiment, the lipotropic agent is betaine hydrochloride.

Suitable polyamines include spermidine, putrescine, spermine, agmatine, cadaverine, and any combinations thereof. In one embodiment, the lipotropic agent is spermidine.

Suitable lysis buffers include surfactants, in particular nonionic surfactants. Specific nonionic surfactants include Triton® X100 and IGEPAL® CA-630, or a combination thereof. Triton™ X100 is available from Sigma Aldrich®, has the generic name polyethylene glycol tea-octylphenyl ether or t-octylphenoxypolyethoxyethanol, and has the formula t-oct-C₆H₄—(OCH₂CH₂)x, where x is 9 or 10. IGEPAL® CA-630 is available from Sigma Aldrich®, has the generic name octylphenoxy poly(ethyleneoxy)ethanol, branched, and has the formula (C₂H₄O)ₙC₁₄H₂₂O.

Importantly, the methods of the present disclosure provide test results that can identify the existence of a BSI and the type of bacteria responsible in a much shorter time than what is currently available. As previously discussed, prior art methods can take 24 to 72 hours, which causes catastrophic effects for the patient—most notably a significant increase in chances of death for every hour that passes. The present methods, by contrast, can provide a result within four hours or less, as discussed in greater detail below. Further, where other prior art methods may be destructive of the bacteria sample, the methods of the present disclosure provide a viable micro-organism sample that can be further analyzed and tested.

As discussed in greater detail below, the detailed methods described herein provide for the isolation of viable micro-organism(s) (i.e. agents that cause the BSI) from a freshly inoculated blood culture sample, a positive blood culture sample and other bodily fluids, for early detection of micro-organism(s). The detection can be conducted with time-lapse digital microscopy and for subsequent downstream testing of isolated micro-organism(s). The various methods allow for multiple downstream analyses of micro-organism(s) isolated from freshly inoculated blood culture sample and positive blood culture samples.

The present disclosure also provides methods for isolating, detecting, and/or evaluating viable micro-organism(s) from a freshly collected blood culture or from a blood culture sample that has tested positive for the presence of micro-organism(s). These methods include obtaining a biological sample determined to contain at least one micro-organism, combining at least a portion of the biological sample with betaine hydrochloride and spermidine-containing lysis reagents to lyse the non-target cells (e.g. blood cells in the blood sample) in the biological sample, isolating the intact micro-organism(s), early detection of micro-organism(s) growth in a biological sample, optionally preparing a plated pure culture or a single inoculum, and performing downstream analysis on the isolated, viable microorganism(s) or optional pure culture/inoculum.

In FIG. 1, a first embodiment of the method of the present disclosure is shown, with reference numeral 1000. A culture is first taken from a patient who is suspected to have a BSI (step 1001). The sample is allowed to incubate for a period of time (e.g., 2-3 hours) at an elevated temperature (e.g. 30° C.-35° C.) with agitation (step 1002). A portion of a freshly inoculated blood culture sample (e.g., 5-10 mL) is obtained from the culture (step 1003). An amount of a lysis reagent (e.g., 0.5-1 mL) is added to the blood culture (step 1004). The reagent is discussed in greater detail below.

The mixture of freshly inoculated blood culture sample and lysis reagent is vortexed for a period of time (e.g. 30-60 seconds), mixed well, and incubated at room temperature for up to five minutes (step 1005), to produce an incubated, lysed sample. The incubated lysed sample is diluted (e.g., 1:10-1:20 dilution) with betaine hydrochloride in water at the final concentration of betaine hydrochloride when added to lysed sample of about 1 millimolar, and mixed (step 1006). The diluted sample is centrifuged (e.g. 2000 g-3000 g) for up to 10 minutes to produce a supernatant and a pellet (step 1007). The pellet will contain the micro-organisms, if any. The supernatant is discarded (step 1007a).

The pellet, containing the isolated and viable microorganism(s), is re-suspended in (e.g., 0.1-0.3 mL) of a growth medium (step 1008). The growth medium is discussed in greater detail below. The re-suspended pellet of isolated/viable microorganism(s) is vortexed and mixed well (step 1008). The re-suspended isolated/viable microorganism(s) is then centrifuged (e.g., at about 150 g-175 g) for a period of time (e.g., up to 10 minutes)(step 1009). The supernatant is transferred to a single well in a well plate (e.g., 96 well plate)(step 1010), while the pellet is discarded (step 1009a). The well plate is centrifuged (e.g., at about 100 g-200 g for up to 5 minutes)(step 1011) and then immediately subjected to time-lapse digital microscopic observations and analysis (step 1012). The sample with positive growth of micro-organism(s) is subjected to Gram stain (step 1013). This helps identify the specific types of microorganisms present in the sample. The total amount of time that the method of FIG. 1 takes can be four hours or less.

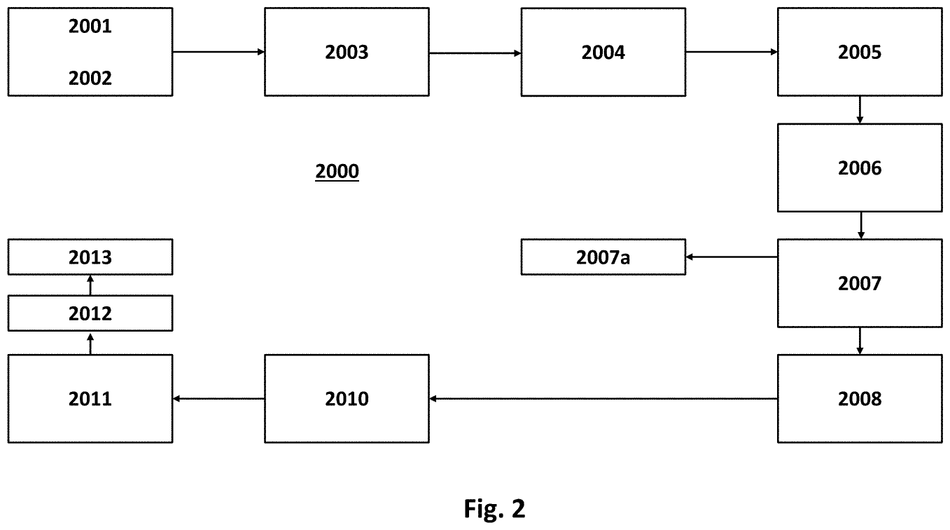

Referring to FIG. 2, a second method of the present disclosure is shown, with reference numeral 2000. Method 2000 is similar to method 1000, with some important differences discussed below. In method 2000, a culture is first taken from a patient who is suspected to have a BSI (step 2001). The sample is allowed to incubate for a period of time (e.g., 2-3 hours) at an elevated temperature (e.g., 30° C.-35° C.) with agitation (step 2002). A portion of a freshly inoculated blood culture sample (e.g., 5-10 mL) is obtained from the culture (step 2003). An amount of a lysis reagent (e.g., 0.5-1 mL) is added to the blood culture portion (step 2004). Again, the reagent is discussed in greater detail below.

The mixture of freshly inoculated blood culture sample and lysis reagent is vortexed for a period of time (e.g., 30-60 seconds), mixed well, and incubated at room temperature for up to five minutes (step 2005), to produce an incubated, lysed sample. The incubated lysed sample is diluted (e.g., 1:10-1:20 dilution) with betaine hydrochloride in water at the final concentration of betaine hydrochloride when added to lysed sample of 0.5-1 millimolar (step 2006). The diluted sample is centrifuged (e.g., at about 2000 g-3000 g) for up to 10 minutes to produce a supernatant and a pellet (step 2007). The pellet will contain the micro-organisms, if any. The supernatant is discarded (step 2007a).

The pellet, containing the isolated and viable microorganism(s), is resuspended in (e.g., 0.1-0.3 mL) of a growth medium (step 2008). The growth medium is discussed in greater detail below. Here, method 2000 differs from method 1000. Rather than another centrifuge step where the resuspended pellet is centrifuged again (as in method 2010), in method 2000 the pellet from step 2008 is transferred directly to a single well in a well plate (e.g., 96 well plate)(step 2010). The well plate is then centrifuged (e.g., at about 200 g for up to 5 minutes)(step 2011) and then immediately subjected to time-lapse digital microscopic observations and analysis (step 2012). The sample with positive growth of micro-organism(s) is subjected to Gram stain (step 2013). This helps identify the specific types of microorganisms present in the sample. The total amount of time that the method of FIG. 2 takes can be three and one half hours or less. Method 2000 has two centrifuge steps, where method 1000 had three.

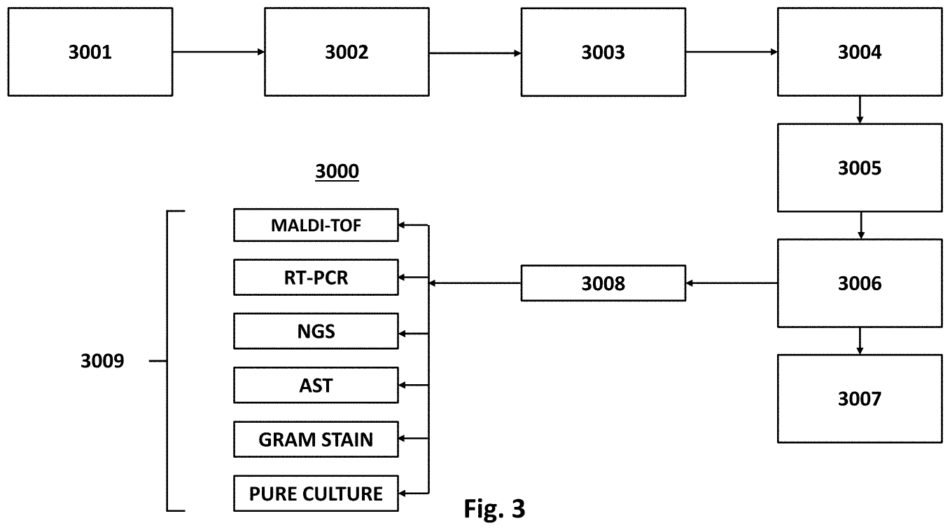

A third method, depicted in FIG. 3 and referenced with numeral 3000, differs from methods 1000 and 2000 in that it is presumed or known that the patient has a BSI (step 3001). Thus, in method 3000, a portion of a positive blood culture (PBC) sample (e.g., 5-10 mL) is obtained (step 3002). A reagent is added to the PBC sample (step 3003). The mixture of PBC sample and lysis reagent is vortexed for a period of time (e.g., 30-60 seconds), mixed well, and incubated at room temperature for a period of time (e.g. up to five minutes)(step 3004). The incubated lysed sample is diluted (e.g., 1:10-1:20 dilution) with betaine hydrochloride in water, so that the final concentration of betaine hydrochloride when added to the lysed sample is 0.5-1 millimolar (step 3005).

The diluted sample is centrifuged (e.g., at about 2000 g-3000 g for up to 10 minutes) to produce supernatant and pellet (step 3006). The supernatant is discarded (step 3007), while the pellet, containing isolated/viable microorganism(s), is retained (step 3008). The pellet can then be subjected to any number of diagnostic tests to determine the type of micro-organism present in the sample (step 3009). For example, these tests may include matrix-assisted laser adsorption ionization time-of-flight mass spectrometry (MALDI-TOF), real-time polymerase chain reaction (RT-PCR), next generation sequencing (NGS), antibiotic susceptibility testing (AST), Gram staining, and pure culture techniques. The total amount of time that the method of FIG. 3 takes can be thirty minutes or less. In method 3000, there is a single centrifuge step.

Table 1 below shows the ingredients and amounts for one embodiment of the lysis reagent composition, which are the molar or by volume amounts of each ingredient after the lysis reagent composition is added to the blood sample. The present disclosure has unexpectedly discovered that the betaine hydrochloride and spermidine provide excellent ability to keep the microorganisms viable after they are extracted from the patient's body and incubated, vortexed, and centrifuged, as described in the methods above. This is critical in that it allows for a myriad of diagnostic tests that can be performed on the sample to determine the types of microorganisms present. The composition of Table 1 may also include the above-identified alternatives, for example oxibetaine for betaine hydrochloride, or putrescine for spermidine.

TABLE 1
LYSIS REAGENT RECIPE FOR RECOVERY
OF MICROORGANISM(S) FROM BLOOD
AND BIOLOGICAL FLUIDS

| CHEMICAL COMPONENT | CONCENTRATION RANGE | |
|---|---|---|
| BETAINE HYDROCHLORIDE | 0.5-1 | mM |
| SPERMIDINE | 0.25-1 | mM |
| TRITON X-100 | 0.2727-0.3636% | by volume |
| SAPONIN | 0.0909-0.2272% | by volume |

Table 2 below shows the composition of the growth medium used in methods 10 and 100.

TABLE 2
GROWTH MEDIUM COMPOSITION

| | AMOUNT | |
|---|---|---|
| COMPONENT | (WEIGHT/VOLUME; W/V) | |
| BEEF HEART (infusion from 250 g) | 4-6 | g/L |
| CALF BRIAN (infusion from 200 g) | 10-14 | g/L |
| Na₂HPO₄ | 1.5-3 | g/L |
| D(+)-GLUCOSE | 1-3 | g/L |
| PEPTONE | 8-12 | g/L |
| NaCl | 4-5 | g/L |
| YEAST EXTRACT | 5-10 | g/L |

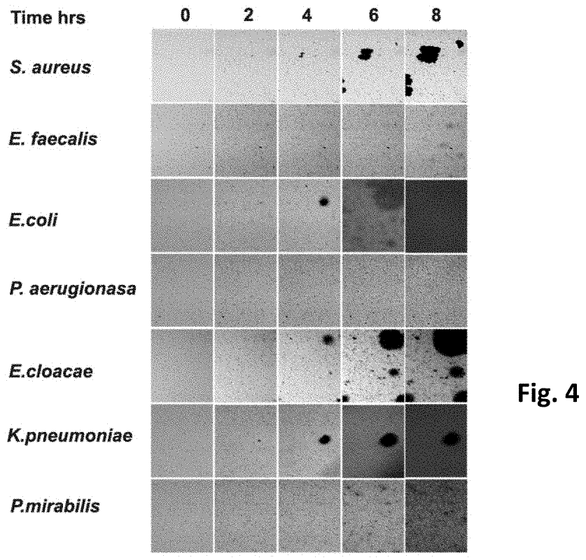

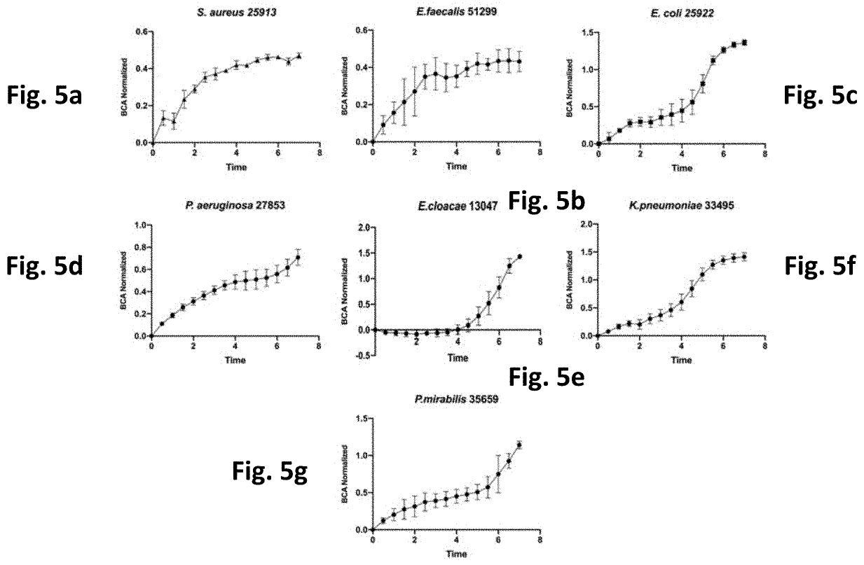

Tables 3 and 4 and FIGS. 4 through 5g relate to the results achieved when the methods of the present disclosure were tested on certain blood samples. To begin, blood samples were spiked with certain types of bacteria in the amounts listed in Table 3. Table 4 illustrates the time needed for various stages of the presently described methods. FIGS. 4 through 5g illustrate this data in graphical form. Some bacteria, for example E. cloacae, may take a longer time to grow than others. However, as seen in Table 4, in all cases, the total time to make a determination of the presence and type of a BSI, was under 8.5 hours. With most of the shown bacteria, the needed time was 6.5 hours or less, or 5.5 hours or less. If the bacterial count is high in the blood sample, then the total time to determine the presence of a BSI can be even less, namely 4 hours or less (as in described previously). If the bacterial count is low, for example the low bacterial counts listed in Table 3, then it takes more time to detect the growth, as indicated by the times in Table 4. In any case, the present disclosure provides a vast improvement over current methods, which as previously discussed can take as long as 24 to 72 hours. The methods and compositions of the present disclosure thus provide significant benefits to patients battling BSI and the medical professionals treating them.

TABLE 3
SPIKING OF BLOOD CULTURE BOTTLE

| QC ORGANISM | SPIKED CFU/mL |
|---|---|
| Escherichia coli 25922 | 11 |
| Enterobacter cloacae 13047 | 12 |
| Enterococcus faecalis 51299 | <1 |
| Klebsiella pneumoniae 33495 | 10 |
| Pseudomonas aeruginosa 27853 | 11 |
| Proteus mirabilis 35659 | <1 |
| Staphylococcus aureus 25913 | 12 |

TABLE 4
TOTAL TIME TO DETECT MICROORGANISM(S) GROWTH

| | INITIAL | | | TOTAL |
|---|---|---|---|---|
| | INCUBA- | PRO- | MICROS- | TIME |
| | TION | CESSING | COPY | TO MAKE |
| QC | TIME | TIME | TIME | A CALL |
| ORGANISM | (hours) | (hours) | (hours) | (hours) |
| Escherichia | 2-3 | 0.5 | 3 | 4.5-5.5 |
| coli 25922 | | | | |
| Enterobacter | 2-3 | 0.5 | 3 | 7.5-−8.5 |
| cloacae 13047 | | | | |
| Enterococcus | 2-3 | 0.5 | 3 | 4.5-5.5 |
| faecalis 51299 | | | | |
| Klebsiella | 2-3 | 0.5 | 3 | 5.5-6.5 |
| pneumoniae | | | | |
| 33495 | | | | |
| Pseudomonas | 2-3 | 0.5 | 3 | 5.5-6.5 |
| aeruginosa | | | | |
| 27853 | | | | |
| Proteus | 2-3 | 0.5 | 3 | 5.5-6.5 |
| mirabilis 35659 | | | | |
| Staphylococcus | 2-3 | 0.5 | 3 | 5.5-6.5 |
| aureus 25913 | | | | |

While the present disclosure has been described with reference to one or more exemplary embodiments, it will be understood by those skilled in the art that various changes may be made and equivalents may be substituted for elements thereof without departing from the scope of the present disclosure. For any ranges described above, such as time, amount, or concentration, the present specification contemplates that range, as well as any subranges therebetween. For example, if the present specification recites a range of 30 to 60 seconds, the present disclosure also contemplates 35-55 seconds, 40-50 seconds, 30-55 seconds, etc. In addition, many modifications may be made to adapt a particular situation or material to the techniques of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A reagent composition for blood lysis, comprising:
a polyamine;
a lipotropic agent;
a saponin; and
a surfactant.

2. The reagent composition of claim 1, wherein the polyamine is selected from the group consisting of spermidine, putrescine, spermine, agmatine, cadaverine, and any combinations thereof.

3. The reagent composition of claim 1, wherein the polyamine is spermidine.

4. The reagent composition of claim 1, wherein the lipotropic agent is selected from the group consisting of betaine hydrochloride, oxibetaine, trimethlyglycine, inositol, methionine, and any combinations thereof.

5. The reagent composition of claim 1, wherein the lipotropic agent is betaine hydrochloride.

6. The reagent composition of claim 1, wherein the surfactant is a nonionic surfactant with a hydrophilic polyethylene oxide chain.

7. The composition of claim 1, wherein the composition comprises:
between 0.25 to 1 millimolar of the polyamine;
between 0.5 to 1 millimolar of the lipotropic agent;
between 0.2272 to 0.3636% by volume of the surfactant; and
between 0.0909 to 0.2272% by volume of the saponin.

8. A method of testing a blood sample of a patient for a blood stream infection that is caused by at least one bacterium, comprising the steps of:
drawing a sample from the patient;
mixing the composition of claim 1 with the sample to form a first mixture;
centrifuging the first mixture to separate the first mixture into a supernatant and a pellet;
discarding the supernatant;
placing the pellet into a growth medium, to form a second mixture;
centrifuging the second mixture; and
testing the second mixture to determine the presence of the at least one bacterium.

9. The method of claim 8, further comprising the step of, after the mixing step and before the first centrifuging step, diluting the first mixture with a second composition that comprises betaine hydrochloride and water.

10. The method of claim 8, further comprising the step of, after the centrifuging the second mixture step and before the testing step, discarding a second pellet created during the centrifuging the second mixture step.

11. The method of claim 8, wherein the first mixture comprises:
between 0.25 to 1 millimolar of the polyamine;
between 0.5 to 1 millimolar of the lipotropic agent;
between 0.2272 to 0.3636% by volume of the surfactant;
and between 0.0909 to 0.2272% by volume of the saponin.

12. A method of testing a blood sample of a patient known to have a blood stream infection that is caused by at least one bacterium, comprising the steps of:
drawing a sample from the patient;
mixing the composition of claim 1 with the sample to form a first mixture;
centrifuging the first mixture to separate the first mixture into a supernatant and a pellet;
discarding the supernatant and retaining the pellet;
testing the pellet to determine the type of the at least one bacterium.

13. The method of claim 12, further comprising the step of, after the mixing step and before the first centrifuging step, diluting the first mixture with a second composition that comprises betaine hydrochloride and water.
